(19) United States
(12) Patent Application Publication
(10) Pub. No.: US 2023/0272329 A1
(43) Pub. Date: Aug. 31, 2023

(54) SUBSTANCE INTRODUCTION DEVICE AND SUBSTANCE INTRODUCTION METHOD

(71) Applicant: TERUMO KABUSHIKI KAISHA, Tokyo (JP)

(72) Inventors: Kodai KITAMURA, Kanagawa (JP); Yuu NAKANO, Kanagawa (JP); Youichi HIGUCHI, Kanagawa (JP)

(21) Appl. No.: 18/081,578

(22) Filed: Dec. 14, 2022

(30) Foreign Application Priority Data
Feb. 28, 2022 (JP) 2022-030397

(51) Int. Cl. C12M 1/42; C12M 1/00; C12N 13/00

(52) U.S. Cl. CPC C12M 35/02; C12M 29/04; C12N 13/00; C12M 45/07

(57) ABSTRACT

A substance introduction device introduces a substance into a cell by electroporation, and includes at least one substance introduction unit including an accommodation container that accommodates a cell suspension containing the cell and the substance and a pair of electrodes that applies a voltage to the cell suspension accommodated in the accommodation container; and a liquid delivery unit that delivers a liquid to the accommodation container. The accommodation container has a first and a second openings, and the liquid delivery unit fills the accommodation container with the cell suspension before voltage application through the first opening and to discharge the cell suspension after the voltage application from the accommodation container through the second opening, and to fill the accommodation container with a cleaning solution through the second opening and to discharge the cleaning solution from the accommodation container through the first opening.

### CROSS-REFERENCE TO RELATED APPLICATIONS

The present application claims the benefit of and priority, under 35 U.S.C. § 119, to Japanese Application Serial No. 2022-030397, filed on Feb. 28, 2022, entitled “Substance Introduction Device and Substance Introduction Method,” the entire disclosure of which is hereby incorporated herein by reference, in its entirety, for all that it teaches and for all purposes.

### BACKGROUND

The present disclosure relates to a substance introduction device and a substance introduction method.

Conventionally, techniques of introducing substances into cells by electroporation are known. For example, PCT Patent Publication No. WO 2021/177185, entitled “Substance Introduction Device and Substance Introduction Method,” discloses an electroporation device capable of repeatedly executing electroporation in the same container.

### BRIEF SUMMARY

In general, in the electroporation, an optimum electroporation execution condition, such as a voltage value of a voltage to be applied or time for the application, is different depending on an environmental factor such as the amount of a cell suspension to be treated, the volume of a container in which the treatment is executed, or the distance between a pair of electrodes to which a voltage is applied. Therefore, for example, even in a case where electroporation execution conditions for a small amount of cells are established with a certain container, it is necessary to reset the execution conditions again with a different container when the electroporation is executed in the different container in order to increase the amount of the cell suspension to be treated.

In this regard, the device disclosed in PCT Patent Publication No. WO 2021/177185 can repeatedly execute the electroporation in the same container without enlarging a container for execution of the electroporation, so that the total amount of a cell suspension to be treated can be increased without changing electroporation execution conditions.

However, there is a demand for further improvement in usefulness of the techniques of introducing substances into cells by the electroporation. For example, it is required not to decrease the substance introduction efficiency in electroporation when the electroporation is repeatedly executed in the same container.

An object of the present disclosure that has been made in view of such circumstances is to provide a substance introduction device and a substance introduction method for improving usefulness of a technique of introducing a substance into a cell by electroporation.

A substance introduction device according to an embodiment of the present disclosure is a substance introduction device, which introduces a substance into a cell by electroporation, and includes: one or more substance introduction units, the substance introduction unit including an accommodation container configured to accommodate a cell suspension containing the cell and the substance and a pair of electrodes configured to apply a voltage to the cell suspension accommodated in the accommodation container; and a liquid delivery unit configured to deliver a liquid to the accommodation container. The accommodation container has a first opening and a second opening, and the liquid delivery unit is configured to fill the accommodation container with the cell suspension before voltage application through the first opening and to discharge the cell suspension after the voltage application from the accommodation container through the second opening, and to fill the accommodation container with a cleaning solution through the second opening and to discharge the cleaning solution from the accommodation container through the first opening.

In the substance introduction device according to the embodiment of the present disclosure, the substance introduction unit preferably includes an opening and closing unit configured to be capable of opening and closing the second opening of the accommodation container.

In the substance introduction device according to the embodiment of the present disclosure, the first opening is preferably configured to be located vertically above the second opening.

It is preferable that the substance introduction device according to the embodiment of the present disclosure include a plurality of substance introduction units, connected in series, as the one or more substance introduction units, and that the plurality of substance introduction units be connected in series by linking a second opening of an accommodation container of a first substance introduction unit and a first opening of an accommodation container of a second substance introduction unit, the first substance introduction unit and the second substance introduction unit being included in the plurality of substance introduction units.

The substance introduction device according to the embodiment of the present disclosure preferably includes a filtering unit configured to remove a cell residue from the cell suspension before the voltage application with which the accommodation container is to be filled.

The substance introduction device according to the embodiment of the present disclosure preferably includes a filtering unit configured to remove a cell residue from the cell suspension after the voltage application discharged from the accommodation container.

The substance introduction device according to the embodiment of the present disclosure preferably includes a cell counter configured to count a living cell in the cell suspension before the voltage application with which the accommodation container is to be filled.

The substance introduction device according to the embodiment of the present disclosure preferably includes a cell counter configured to count a living cell in the cell suspension after the voltage application discharged from the accommodation container.

A substance introduction method according to an embodiment of the present disclosure is a substance introduction method for introducing a substance into a cell by electroporation, the substance introduction method including: preparing one or more substance introduction units, the substance introduction unit including an accommodation container configured to accommodate a cell suspension containing the cell and the substance and a pair of electrodes configured to apply a voltage to the cell suspension accommodated in the accommodation container; filling the accommodation container with the cell suspension before voltage application through a first opening of the accommodation container; applying, by the pair of electrodes, a voltage to the cell suspension accommodated in the accommodation container; discharging the cell suspension after the voltage application from the accommodation container through a second opening of the accommodation container; filling the accommodation container with a cleaning solution through the second opening; and discharging the cleaning solution from the accommodation container through the first opening.

In the substance introduction method according to the embodiment of the present disclosure, it is preferable that the first opening be configured to be located vertically above the second opening, that the filling the accommodation container with the cleaning solution through the second opening include filling the accommodation container with the cleaning solution from a lower side of the accommodation container through the second opening, and that the discharging the cleaning solution from the accommodation container through the second opening include discharging the cleaning solution from an upper side of the accommodation container through the first opening.

The substance introduction method according to the embodiment of the present disclosure preferably further includes removing a cell residue from the cell suspension before the voltage application with which the accommodation container is to be filled.

The substance introduction method according to the embodiment of the present disclosure preferably further includes removing a cell residue from the cell suspension after the voltage application discharged from the accommodation container.

The substance introduction method according to the embodiment of the present disclosure preferably further includes counting a living cell in the cell suspension before the voltage application with which the accommodation container is to be filled.

The substance introduction method according to the embodiment of the present disclosure preferably further includes counting a living cell in the cell suspension after the voltage application discharged from the accommodation container.

The substance introduction device and the substance introduction method according to the present disclosure can improve the usefulness of the technique of introducing the substance into the cell by the electroporation.

### BRIEF DESCRIPTION OF THE SEVERAL VIEWS OF THE DRAWINGS

FIG. 1 is a schematic diagram schematically illustrating a schematic configuration of a substance introduction device according to a first embodiment using a partial cross-sectional view;

FIG. 2 is a flowchart illustrating an operation of the substance introduction device according to the first embodiment; and

FIG. 3 is a schematic diagram illustrating a schematic configuration of a substance introduction device according to a second embodiment.

### DETAILED DESCRIPTION

Hereinafter, a substance introduction device 1 according to an embodiment of the present disclosure will be described with reference to the drawings.

In the drawings, the same or equivalent portions are denoted by the same reference signs. In the description of the embodiment, the description regarding the same or equivalent portions is omitted or simplified as appropriate.

The substance introduction device 1 according to the embodiment of the present disclosure introduces a substance into a cell by electroporation. Electroporation is a method in which an electrical pulse or the like is applied to a cell suspension containing a cell and a substance to be introduced into the cell to perforate a cell membrane of the cell and introduce the substance into the cell. Electroporation is also referred to as an electroporation method or a process of electroporation. In the embodiment of the present disclosure, the cell suspension is obtained by suspending, for example, an animal cell or a cell such as Escherichia coli, and a substance such as a gene, deoxyribonucleic acid (DNA), ribonucleic acid (RNA), nucleic acid, protein, a low molecular weight compound, a lipid molecule, or a liposome in a buffer solution (buffer).

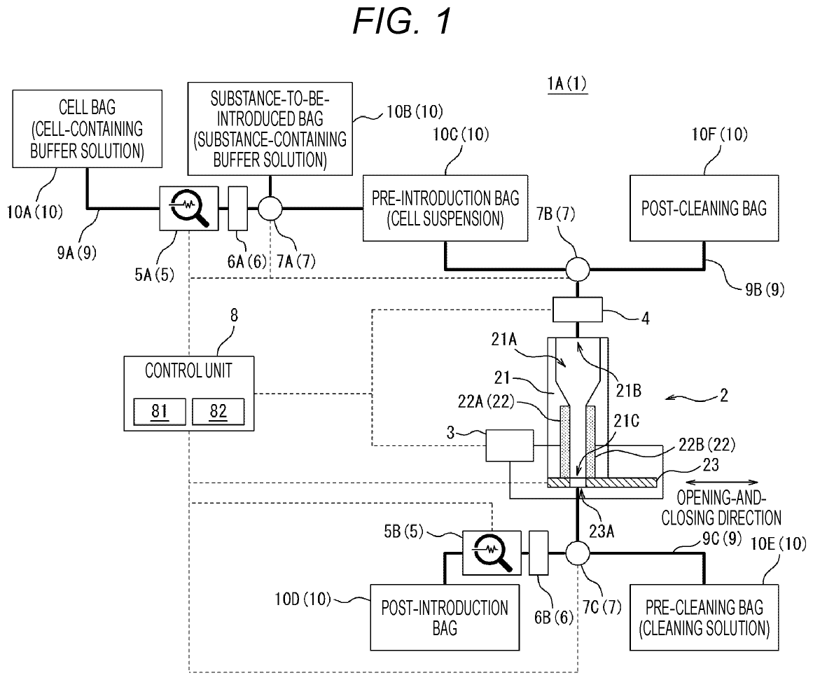

A substance introduction device 1A, which is a first embodiment of the substance introduction device 1 according to the present disclosure, will be described with reference to FIG. 1. FIG. 1 is a schematic diagram schematically illustrating a schematic configuration of the substance introduction device 1A according to the first embodiment of the present disclosure using a partial cross-sectional view. As illustrated in FIG. 1, the substance introduction device 1A includes a substance introduction unit 2, a voltage application unit 3, a liquid delivery unit 4, a cell counter 5, a filtering unit 6, a valve unit 7, and a control unit 8.

As will be described later, a plurality of bags 10 are attached to the substance introduction device 1A via liquid delivery paths 9 such as liquid delivery tubes. In this embodiment, a cell bag 10A, a substance-to-be-introduced bag 10B, a pre-introduction bag 10C, a post-introduction bag 10D, a pre-cleaning bag 10E, and a post-cleaning bag 10F are attached to the substance introduction device 1A as illustrated in FIG. 1. The cell bag 10A accommodates cells together with a buffer solution. The substance-to-be-introduced bag 10B accommodates a substance to be introduced into the cells together with a buffer solution. The pre-introduction bag 10C accommodates a cell suspension before substance introduction by electroporation. The post-introduction bag 10D accommodates a cell suspension into which the substance has been introduced by electroporation. The pre-cleaning bag 10E accommodates a cleaning solution to be used for cleaning of the substance introduction unit 2. The post-cleaning bag 10F accommodates the cleaning solution after cleaning the substance introduction unit 2.

As illustrated in FIG. 1, the cell bag 10A and the substance-to-be-introduced bag 10B are connected to the pre-introduction bag 10C by a liquid delivery path 9A in the substance introduction device 1A. The pre-introduction bag 10C and the post-cleaning bag 10F are connected to a first opening 21B of the substance introduction unit 2 by a liquid delivery path 9B. The post-introduction bag 10D and the pre-cleaning bag 10E are connected to a second opening 21C of the substance introduction unit 2 by a liquid delivery path 9C.

In the embodiment, the inside of the substance introduction unit 2 is cleaned by a cleaning solution after execution of the electroporation process. This cleaning removes stains, such as cell residues or air bubbles, adhering to the inside of the substance introduction unit 2, particularly, electrode surfaces of a pair of electrodes 22. The cell residues include, for example, a dead cell, a damaged cell (fracture), protein, nucleic acid, or the like. Since the inside of the substance introduction unit 2 is cleaned after execution of the electroporation process as described above, the substance introduction efficiency in the electroporation is less likely to decrease when the electroporation is repeatedly executed in the same container. The cleaning solution is, for example, a buffer solution, but is not limited thereto.

The substance introduction unit 2 is an instrument that is used to introduce the substance into the cell by electroporation. In FIG. 1, the substance introduction unit 2 is illustrated in a cross-sectional view. The substance introduction unit 2 includes an accommodation container 21, the pair of electrodes 22, and an opening and closing unit 23.

The accommodation container 21 is configured to accommodate the cell suspension containing the cell and the substance to be introduced into the cell. The accommodation container 21 is, for example, a cuvette. The accommodation container 21 may be made of, for example, glass, plastic, silicon, quartz, or the like. The accommodation container 21 internally partitions an internal space 21A that is long and can accommodate the cell suspension. A size of the internal space 21A may be determined depending on the amount of the cell suspension treated in one electroporation, and is, for example, 10 microliters to 10 milliliters, desirably 40 microliters to 400 microliters.

The accommodation container 21 is a cylindrical container having the first opening 21B and the second opening 21C. The internal spaces 21A partitioned inside the accommodation container 21 communicate with the outside of the accommodation container 21 through the first opening 21B and the second opening 21C, respectively. The first opening 21B is provided on one end side of the accommodation container 21 in a longitudinal direction, and the second opening 21C is provided on the other end side opposite to the first opening 21B of the accommodation container 21 in the longitudinal direction. Thus, the accommodation container 21 is configured such that a liquid that has been charged into the internal space 21A through one of the first opening 21B and the second opening 21C can be discharged to the outside through the other of the first opening 21B and the second opening 21C.

The pair of electrodes 22 is configured to apply a voltage to the cell suspension accommodated in the accommodation container 21. In the embodiment, the pair of electrodes 22 includes a first electrode 22A and a second electrode 22B. Both the first electrode 22A and the second electrode 22B are plate-shaped electrodes and are made of an energizable (e.g., capable of being electrically energized) material such as metal or a conductive polymer. In the embodiment, the first electrode 22A is assumed to be an anode, and the second electrode 22B is assumed to be a cathode. The first electrode 22A and the second electrode 22B are attached to the accommodation container 21 so as to face each other with the internal space 21A of the accommodation container 21 sandwiched therebetween. The pair of electrodes 22 generates an electrical pulse in the internal space 21A of the accommodation container 21 sandwiched between the pair of electrodes 22 when power is applied from the voltage application unit 3 in a state where the cell suspension is accommodated in the accommodation container 21. Thus, the electroporation is executed in the accommodation container 21 so that the substance is introduced into the cell contained in the cell suspension. A distance between the electrodes constituting the pair of electrodes 22 is set to a distance suitable for executing the electroporation, and is, for example, 0.1 mm to 1 cm, and is desirably in the range of 1 mm to 5 mm.

The pair of electrodes 22 is provided on a side surface of the accommodation container 21 sandwiched between the first opening 21B and the second opening 21C in the longitudinal direction of the accommodation container 21. In this embodiment, the first electrode 22A and the second electrode 22B are attached to the accommodation container 21 such that the respective electrode surfaces are exposed to the internal space 21A of the accommodation container 21. However, the electrode surfaces of the first electrode 22A and the second electrode 22B are not necessarily exposed to the internal space 21A.

The pair of electrodes 22 may be configured to be detachable from the accommodation container 21. Alternatively, the accommodation container 21 and the pair of electrodes 22 may be integrated as cuvette electrodes.

The opening and closing unit 23 is configured to be capable of opening and closing the opening of the accommodation container 21. In the embodiment, the opening and closing unit 23 is attached to the accommodation container 21 so as to cover an end portion of the accommodation container 21 on the second opening 21C side. Thus, the opening and closing unit 23 is configured to be capable of opening and closing the second opening 21C of the accommodation container 21. For example, as illustrated in FIG. 1, the opening and closing unit 23 is a plate-shaped member that has a through-hole 23A and is slidable in an opening-and-closing direction relative to the accommodation container 21. The liquid delivery path 9C may be attached to the through-hole 23A of the opening and closing unit 23 by fitting or the like. The opening and closing unit 23 is slid (e.g., slidably moved) to align a position of the through-hole 23A with a position of the second opening 21C of the accommodation container 21, whereby the opening and closing unit 23 opens the second opening 21C of the accommodation container 21. The opening and closing unit 23 is slid (e.g., slidably moved) to cause the position of the through-hole 23A to be misaligned (e.g., arranged out of alignment) from the position of the second opening 21C of the accommodation container 21, whereby the opening and closing unit 23 closes the second opening 21C of the accommodation container 21.

In the accommodation container 21 of the substance introduction unit 2, the first opening 21B may be configured to be located vertically above the second opening 21C. Thus, when the movement of the cell suspension by gravity is utilized, the cell suspension can be accommodated in the internal space 21A of the accommodation container 21 or can be discharged from the internal space 21A by opening or closing the second opening 21C.

In the embodiment, the opening and closing unit 23 is configured to be able to automatically open and close the opening of the accommodation container 21 under the control of the control unit 8. For example, the opening and closing unit 23 may include a motor for sliding the opening and closing unit 23 itself relative to the accommodation container 21. However, the opening and closing unit 23 may be configured to be able to manually open and close the opening of the accommodation container 21. Further, the opening and closing unit 23 is not limited to the above-described configuration, and may be any instrument capable of automatically or manually opening and closing the opening of the accommodation container 21, such as an opening and closing valve, a lid, or a door. The substance introduction unit 2 may further include an opening and closing unit 23 configured to be capable of opening and closing the first opening 21B, in addition to the opening and closing unit 23 configured to be capable of opening and closing the second opening 21C of the accommodation container 21.

The substance introduction unit 2 is detachable from the substance introduction device 1A. Thus, the substance introduction unit 2 can be replaced with a new substance introduction unit 2 in the case of being damaged, degraded, or the like. Alternatively, the substance introduction units 2 respectively having different volumes of the accommodation containers 21 can be attached to the substance introduction device 1A in accordance with the amount of the cell suspension to be treated in one electroporation. Furthermore, a plurality of the substance introduction units 2 connected in series may be attached to the substance introduction device 1A as will be described later. However, the substance introduction unit 2 may be integrated with the substance introduction device 1A or is not necessarily detachable from the substance introduction device 1A.

The voltage application unit 3 is an instrument that applies a voltage between the pair of electrodes 22. The voltage application unit 3 is not particularly limited as long as a voltage can be applied between the pair of electrodes 22. The voltage application unit 3 may include, for example, a storage battery or a dry battery. Further, for example, the voltage application unit 3 may include an adapter or the like for receiving power supply from an external power source to receive the power supply from the external power source. The voltage application unit 3 is connected to each of the pair of electrodes 22 by a conductive wire or the like so as to be capable of supplying power as indicated by a solid line in FIG. 1. Thus, the voltage application unit 3 applies a predetermined voltage between the pair of electrodes 22, so that the electroporation is executed in the accommodation container 21, and the substance is introduced into the cells contained in the cell suspension. In the embodiment, it is assumed that the first electrode 22A serves as the anode and the second electrode 22B serves as the cathode when the voltage is applied by the voltage application unit 3.

The voltage application unit 3 may include one or more sensors. The sensor is, for example, an ohmmeter, a voltmeter, an ammeter, or the like. Thus, the voltage application unit 3 can measure a resistance value, a voltage value, or a current value between the pair of electrodes 22 by the sensor, and adjust the voltage applied between the pair of electrodes 22.

The liquid delivery unit 4 includes one or more liquid delivery pumps. The liquid delivery pump is, for example, a syringe-type, suction-type, or peristaltic pump, but is not limited thereto. The liquid delivery unit 4 is configured to be automatically operable under the control of the control unit 8, but may be configured to be manually operable. In the embodiment, the liquid delivery unit 4 is provided in the liquid delivery path 9B connected to the first opening 21B of the accommodation container 21 of the substance introduction unit 2. Thus, the liquid delivery unit 4 can deliver a liquid, such as the cell suspension or the cleaning solution, to the internal space 21A of the accommodation container 21. However, the liquid delivery unit 4 may be provided at any position of the liquid delivery path 9 included in the substance introduction device 1A.

The liquid delivery unit 4 fills the accommodation container 21 with the cell suspension before voltage application through the first opening 21B of the accommodation container 21, and discharges the cell suspension after voltage application from the accommodation container 21 through the second opening 21C. Further, the liquid delivery unit 4 fills the accommodation container 21 with the cleaning solution through the second opening 21C of the accommodation container 21, and discharges the cleaning solution from the accommodation container 21 through the first opening 21B. As described above, a direction in which the cleaning solution flows inside the accommodation container 21 of the substance introduction unit 2 is set to be opposite to a direction in which the cell suspension flows in the accommodation container 21 of the substance introduction unit 2, so that the stains, such as the cell residues or air bubbles, are likely to be peeled off from the accommodation container 21, and an effect of cleaning the pair of electrodes 22 and the accommodation container 21 and the pair of electrodes 22 can be improved. In particular, in a case where the substance introduction unit 2 is arranged such that the first opening 21B is higher than the second opening 21C, the cell residues or the like are likely to adhere to the lower side (that is, the second opening 21C side in the embodiment) of the accommodation container 21 by gravity, and thus, it is effective to cause the cleaning solution to flow from the second opening 21C side.

The cell counter 5 is configured to count living cells. The living cell is a cell which is not dead or damaged and into which the substance can be introduced by electroporation. The cell counter 5 includes, for example, equipment such as a flow cytometer, a particle size distribution meter, and a micropore device. In this embodiment, the substance introduction device 1A includes a cell counter 5A provided in the liquid delivery path 9A and a cell counter 5B provided in the liquid delivery path 9C. However, the cell counter 5 may be provided at any position of the liquid delivery path 9 included in the substance introduction device 1A.

The cell counter 5A is configured to count living cells in the cell suspension before voltage application with which the accommodation container 21 is to be filled. Thus, quantities or ratios of the cells contained in the cell suspension and the substance to be introduced into the cells can be appropriately set by adjusting the quantity of the substance to be mixed in the cell suspension in accordance with the number of the living cells, and the substance introduction efficiency in the electroporation can be improved.

The cell counter 5B is configured to count living cells in the cell suspension after voltage application discharged from the accommodation container 21. Thus, it is possible to grasp the number of living cells into which the substance has been introduced without dying or being damaged by electroporation. Furthermore, the number of the cell residues remaining in the accommodation container 21 of the substance introduction unit 2 can be estimated based on the number of the living cells.

The filtering unit 6 is configured to remove the cell residues. As described above, the cell residues include the dead or damaged cell, the protein, the nucleic acid, or the like. The filtering unit 6 includes, for example, magnetic beads, nonwoven fabric, an electret filter (electrostatic filter material), or the like. In this embodiment, the substance introduction device 1A includes a filtering unit 6A provided in the liquid delivery path 9A and a filtering unit 6B provided in the liquid delivery path 9C. However, the filtering unit 6 may be provided at any position of the liquid delivery path 9 included in the substance introduction device 1A.

The filtering unit 6A is configured to remove cell residues from the cell suspension before voltage application with which the accommodation container 21 is to be filled. Thus, the cell residues contained in the cell suspension are reduced, and the substance introduction efficiency in the electroporation can be improved. Further, since the cell residues are removed in advance from the cell suspension with which the accommodation container 21 of the substance introduction unit 2 is to be filled, the accommodation container 21 and the pair of electrodes 22 are less likely to be contaminated, and the substance introduction efficiency in the electroporation can be further improved.

The filtering unit 6B is configured to remove cell residues from the cell suspension after voltage application discharged from the accommodation container 21. This makes it easy to recover the living cell into which the substance has been introduced by the electroporation.

The valve unit 7 is configured to be capable of opening and closing the liquid delivery path 9 or capable of switching a flow path of the liquid delivery path 9. The valve unit 7 includes, for example, an electromagnetic valve and the like, and is configured to be automatically operable under the control of the control unit 8. However, the valve unit 7 may be configured to be manually operable.

The control unit 8 includes a memory 81 and a processor 82. As the control unit 8, for example, a computer, such as an electroporator, a personal computer, a smartphone, or a tablet terminal, may be used.

The memory 81 is, for example, a semiconductor memory, a magnetic memory, an optical memory, or the like. The memory 81 functions as, for example, a main storage device, an auxiliary storage device, or a cache memory. The memory 81 stores any information to be used for an operation of the substance introduction device 1. For example, the memory 81 stores a system program, an application program, embedded software, or the like.

The processor 82 may be a general-purpose processor, such as a central processing unit (CPU), or a dedicated processor specialized for specific processing. The processor 82 may include, for example, a dedicated circuit such as a field-programmable gate array (FPGA) or an application-specific integrated circuit (ASIC).

As indicated by a broken line in FIG. 1, the control unit 8 is connected to each of the components of the substance introduction device 1A such as the opening and closing unit 23, the voltage application unit 3, the liquid delivery unit 4, the cell counter 5, and the valve unit 7 of the substance introduction unit 2 so as to be capable of communicating therewith in a wired or wireless manner. As a result, the control unit 8 controls these components in order to implement functions of the substance introduction device 1A.

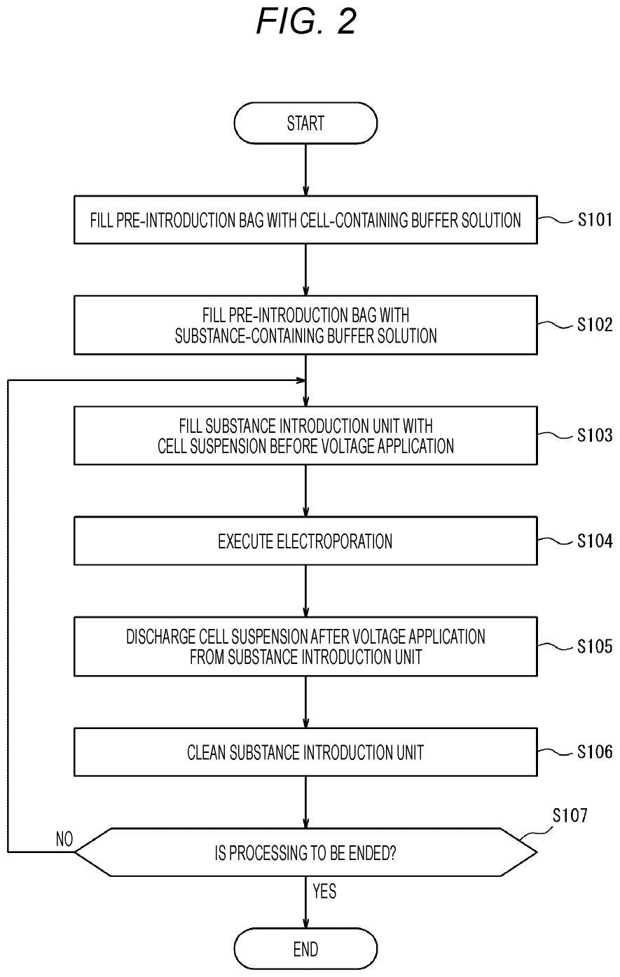

The operation of the substance introduction device 1A according to this embodiment will be described with reference to FIG. 2. FIG. 2 is a flowchart illustrating an operation of the substance introduction device 1A according to the first embodiment. This operation corresponds to a substance introduction method that is executed by the substance introduction device 1A according to this embodiment. In execution of the operation, the above-described substance introduction unit 2 is prepared in advance.

In steps S101 and S102, the control unit 8 prepares the cell suspension before substance introduction by electroporation.

Step S101: The control unit 8 fills the pre-introduction bag 10C with the cell-containing buffer solution from the cell bag 10A.

In the embodiment, the control unit 8 controls the valve unit 7A to fill the pre-introduction bag 10C with a predetermined amount of the cell-containing buffer solution from the cell bag 10A via the liquid delivery path 9A. At this time, since the filtering unit 6A is provided in the liquid delivery path 9A, the filtering unit 6A removes cell residues from the cell suspension before voltage application. Further, the control unit 8 controls the cell counter 5A to count the number of living cells present in the cell-containing buffer solution at the time of filling the pre-introduction bag 10C with the cell-containing buffer solution. The control unit 8 may adjust the amount of the cell-containing buffer solution to be charged into the pre-introduction bag 10C from the cell bag 10A in accordance with the number of the living cells counted by the cell counter 5A.

Step S102: The control unit 8 fills the pre-introduction bag 10C with the substance-containing buffer solution from the substance-to-be-introduced bag 10B.

In the embodiment, the control unit 8 controls the valve unit 7A to fill the pre-introduction bag 10C with the substance-containing buffer solution from the substance-to-be-introduced bag 10B via the liquid delivery path 9A. At this time, the control unit 8 adjusts the amount of the substance-containing buffer solution to be charged into the pre-introduction bag 10C from the substance-to-be-introduced bag 10B in accordance with the number of the living cells counted in step S101. However, the control unit 8 may fill the pre-introduction bag 10C with a predetermined amount of the substance-containing buffer solution from the substance-to-be-introduced bag 10B.

Through steps S101 and S102, the cell-containing buffer solution and the substance-containing buffer solution are suspended in the pre-introduction bag 10C, and the cell suspension before substance introduction by electroporation is prepared. However, the processing in steps S101 and S102 may be omitted by preparing the pre-introduction bag 10C in which the cell suspension before substance introduction by electroporation is accommodated in advance.

In steps S103 to S105, the control unit 8 introduces the substance into the cells by the electroporation.

Step S103: The control unit 8 fills the substance introduction unit 2 with the cell suspension before voltage application.

Specifically, the control unit 8 controls the valve unit 7B to connect the pre-introduction bag 10C and the first opening 21B of the accommodation container 21 by the liquid delivery path 9B. Then, the control unit 8 controls the opening and closing unit 23 of the substance introduction unit 2 to close the second opening 21C of the accommodation container 21. Thereafter, the control unit 8 controls the liquid delivery unit 4 to fill the accommodation container 21 with the cell suspension before voltage application from the pre-introduction bag 10 through the first opening 21B of the accommodation container 21. At this time, the control unit 8 may control the liquid delivery unit 4 so as to fill the accommodation container 21 with a predetermined amount of the cell suspension.

Step S104: The control unit 8 executes the electroporation.

Specifically, the control unit 8 controls the voltage application unit 3 to apply power to the pair of electrodes 22 to generate an electrical pulse in the internal space 21A of the accommodation container 21. Thus, the control unit 8 applies a voltage to the cell suspension accommodated in the accommodation container 21 by the pair of electrodes 22.

Step S105: The control unit 8 discharges the cell suspension after voltage application from the substance introduction unit 2.

Specifically, the control unit 8 controls the valve unit 7C to connect the second opening 21C of the accommodation container 21 and the post-introduction bag 10D by the liquid delivery path 9C. Then, the control unit 8 controls the opening and closing unit 23 of the substance introduction unit 2 to open the second opening 21C of the accommodation container 21. Thereafter, the control unit 8 controls the liquid delivery unit 4 to discharge the cell suspension after voltage application from the accommodation container 21 to the post-introduction bag 10D through the second opening 21C of the accommodation container 21. However, the control unit 8 may discharge the cell suspension after voltage application from the accommodation container 21 to the post-introduction bag 10D through the second opening 21C of the accommodation container 21 by its own weight without controlling the liquid delivery unit 4 from the viewpoint of energy saving.

At this time, since the filtering unit 6B is provided in the liquid delivery path 9C, the filtering unit 6B removes cell residues from the cell suspension after voltage application. Further, the control unit 8 controls the cell counter 5B to count the number of living cells present in the cell suspension after voltage application at the time of discharging the cell suspension after voltage application to the post-introduction bag 10D.

Step S106: The control unit 8 executes cleaning of the substance introduction unit 2.

Specifically, the control unit 8 controls the valve unit 7B to connect the post-cleaning bag 10F and the first opening 21B of the accommodation container 21 by the liquid delivery path 9B. Then, the control unit 8 controls the valve unit 7C to connect the pre-cleaning bag 10E and the second opening 21C of the accommodation container 21 by the liquid delivery path 9C. Further, the control unit 8 controls the opening and closing unit 23 of the substance introduction unit 2 to open the second opening 21C of the accommodation container 21.

Thereafter, the control unit 8 controls the liquid delivery unit 4 to fill the accommodation container 21 with the cleaning solution from the pre-cleaning bag 10E through the second opening 21C of the accommodation container 21. Then, the control unit 8 controls the liquid delivery unit 4 to discharge the cleaning solution from the accommodation container 21 to the post-cleaning bag 10F through the first opening 21B. At this time, the control unit 8 may control the liquid delivery unit 4 so as to fill the accommodation container 21 with a predetermined amount of the cleaning solution.

Step S107: The control unit 8 determines whether or not to end the present processing.

Specifically, the control unit 8 determines whether or not the present processing has been repeated a predetermined number of times. The predetermined number of times may be stored in the memory 81 in advance. When it is determined that the processing has not been repeated the predetermined number of times (“NO” in step S107), the control unit 8 repeatedly executes the processing from step S103. On the other hand, when it is determined that the present processing has been repeated the predetermined number of times (“YES” in step S107), the control unit 8 ends the present processing. The control unit 8 may determine whether or not a total amount of the cell suspension subjected to the present processing has reached a predetermined amount instead of determining whether or not the present processing has been repeated the predetermined number of times.

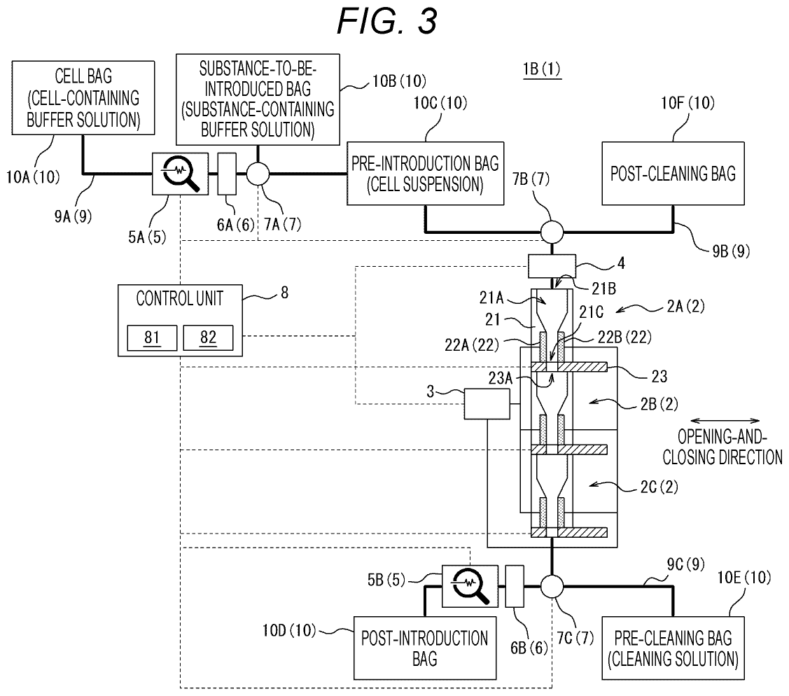

A substance introduction device 1B, which is a second embodiment of the substance introduction device 1 according to the present disclosure, will be described with reference to FIG. 3. FIG. 3 is a schematic diagram illustrating a schematic configuration of the substance introduction device 1B according to the second embodiment.

The second embodiment is different from the first embodiment in terms that a plurality of the substance introduction units 2 (three substance introduction units 2A, 2B, and 2C in this embodiment) are connected in series in the substance introduction device 1B. Hereinafter, the second embodiment will be described focusing on the difference from the first embodiment. Note that portions having the same configurations as those of the first embodiment are denoted by the same reference signs. Hereinafter, the three substance introduction units 2A, 2B, and 2C are collectively referred to simply as the substance introduction units 2 when not particularly distinguished.

As illustrated in FIG. 3, the substance introduction device 1B includes the plurality of substance introduction units 2, the voltage application unit 3, the liquid delivery unit 4, the cell counter 5, the filtering unit 6, the valve unit 7, and the control unit 8.

The plurality of substance introduction units 2 are connected in series. Specifically, the plurality of substance introduction units 2 are connected in series by linking the second opening 21C of an accommodation container 21 of the first substance introduction unit 2 and the first opening 21B of the accommodation container 21 of the second substance introduction unit 2 included in the plurality of substance introduction units 2.

As illustrated in FIG. 3, in the three substance introduction units 2A, 2B, and 2C, the second opening 21C of the accommodation container 21 of the substance introduction unit 2A and the first opening 21B of the accommodation container 21 of the substance introduction unit 2B are linked via the opening and closing unit 23 of the substance introduction unit 2A in this embodiment. Then, the second opening 21C of the accommodation container 21 of the substance introduction unit 2B and the first opening 21B of the accommodation container 21 of the substance introduction unit 2C are linked via the opening and closing unit 23 of the substance introduction unit 2B. Furthermore, the pre-introduction bag 10C and the post-cleaning bag 10F are connected to the first opening 21B of the substance introduction unit 2A by the liquid delivery path 9B. The post-introduction bag 10D and the pre-cleaning bag 10E are connected to the second opening 21C of the substance introduction unit 2C by the liquid delivery path 9C.

In each of the substance introduction units 2A, 2B, and 2C, the pair of electrodes 22 is connected to the voltage application unit 3 by a conductive wire or the like so as to be capable of supplying power. Further, the opening and closing unit 23 in each of the substance introduction units 2A, 2B, and 2C is configured to be capable of automatically opening and closing the opening of the accommodation container 21 under the control of the control unit 8. However, the opening and closing unit 23 may be configured to be able to manually open and close the opening of the accommodation container 21.

As described above, the electroporation can be executed using a plurality of the same containers without enlarging the container for execution of the electroporation, so that a total amount of the cell suspension to be treated can be increased without changing electroporation execution conditions in the individual containers.

Further, since the plurality of substance introduction units 2 are connected in series, it is possible to collectively perform charging or discharging of the cell suspension or the cleaning solution into or from each of the substance introduction units 2. Therefore, the total amount of the cell suspension to be treated can be changed without significantly changing the configuration of the substance introduction device 1B, and the usefulness of the substance introduction device 1B is improved. However, the number of the substance introduction units 2 included in the plurality of substance introduction units 2 is not limited to three, and may be any number of two or more.

An operation of the substance introduction device 1B according to the second embodiment will be described focusing on the difference from the first embodiment with reference to FIG. 2. This operation corresponds to a substance introduction method that is executed by the substance introduction device 1B according to this embodiment. In execution of the operation, the above-described substance introduction unit 2 is prepared in advance.

Step S101: The control unit 8 fills the pre-introduction bag 10C with the cell-containing buffer solution from the cell bag 10A as in the first embodiment.

Step S102: The control unit 8 fills the pre-introduction bag 10C with the substance-containing buffer solution from the substance-to-be-introduced bag 10B as in the first embodiment.

Step S103: The control unit 8 fills the plurality of substance introduction units 2 with the cell suspension before voltage application.

Specifically, the control unit 8 controls the valve unit 7B to connect the pre-introduction bag 10C and the first opening 21B of the accommodation container 21 of the substance introduction unit 2A by the liquid delivery path 9B. Then, the control unit 8 controls the opening and closing units 23 of the substance introduction units 2A, 2B, and 2C to open the second openings 21C of the accommodation containers 21 of the substance introduction units 2A and 2B and to close only the second opening 21C of the accommodation container 21 of the substance introduction unit 2C. Thereafter, the control unit 8 controls the liquid delivery unit 4 to fill the accommodation container 21 of the substance introduction unit 2C with the cell suspension before voltage application from the pre-introduction bag 10. Next, the control unit 8 controls the opening and closing units 23 of the substance introduction units 2A, 2B, and 2C to open the second opening 21C of the accommodation container 21 of the substance introduction unit 2A and to close the second openings 21C of the accommodation containers 21 of the substance introduction units 2B and 2C. Then, the control unit 8 controls the liquid delivery unit 4 to fill the accommodation container 21 of the substance introduction unit 2B with the cell suspension before voltage application from the pre-introduction bag 10. Finally, the control unit 8 controls the opening and closing units 23 of the substance introduction units 2A, 2B, and 2C to close the second openings 21C of all the accommodation containers 21 of the substance introduction units 2A, 2B, and 2C. Then, the control unit 8 controls the liquid delivery unit 4 to fill the accommodation container 21 of the substance introduction unit 2A with the cell suspension before voltage application from the pre-introduction bag 10. As described above, the control unit 8 sequentially fills each of the plurality of substance introduction units 2 with the cell suspension before voltage application in this embodiment.

Step S104: The control unit 8 executes the electroporation.

In this embodiment, the control unit 8 controls the voltage application unit 3 to apply power to each of the pair of electrodes 22 of the plurality of substance introduction units 2, thereby generating an electrical pulse in the internal space 21A of the accommodation container 21 of each of the substance introduction units 2. Thus, the control unit 8 applies the voltage to the cell suspension accommodated in each of the accommodation containers 21 of the plurality of substance introduction units 2.

Step S105: The control unit 8 discharges the cell suspension after voltage application from the plurality of substance introduction units 2.

Specifically, the control unit 8 controls the valve unit 7C to connect the second opening 21C of the accommodation container 21 of the substance introduction unit 2C and the post-introduction bag 10D by the liquid delivery path 9C. Then, the control unit 8 controls the opening and closing unit 23 of each of the plurality of substance introduction units 2 to open the second opening 21C of the accommodation container 21 of each of the substance introduction units 2. At this time, the control unit 8 may control the opening and closing units 23 of the plurality of substance introduction units 2 at once to discharge the cell suspensions after voltage application from the accommodation containers 21 of the plurality of substance introduction units 2 to the post-introduction bag 10D at once. Alternatively, the control unit 8 may sequentially control the opening and closing unit 23 of each of the plurality of substance introduction units 2 to discharge the cell suspension after voltage application from the accommodation containers 21 to the post-introduction bag 10D in the order of, for example, the substance introduction units 2C, 2B, and 2A.

Step S106: The control unit 8 executes cleaning of the plurality of substance introduction units 2.

Specifically, the control unit 8 controls the valve unit 7B to connect the post-cleaning bag 10F and the first opening 21B of the accommodation container 21 of the substance introduction unit 2A by the liquid delivery path 9B. Then, the control unit 8 controls the valve unit 7C to connect the pre-cleaning bag 10E and the second opening 21C of the accommodation container 21 of the substance introduction unit 2C by the liquid delivery path 9C. Furthermore, the control unit 8 controls the opening and closing unit 23 of each of the substance introduction units 2A, 2B, and 2C to open the second opening 21C of the accommodation container 21 of each of the substance introduction units 2A, 2B, and 2C.

Thereafter, the control unit 8 controls the liquid delivery unit 4 to fill the accommodation containers 21 of the substance introduction units 2A, 2B, and 2C with the cleaning solution from the pre-cleaning bag 10E. Then, the control unit 8 controls the liquid delivery unit 4 to discharge the cleaning solution from the accommodation containers 21 of the substance introduction units 2A, 2B, and 2C to the post-cleaning bag 10F.

Step S107: The control unit 8 determines whether or not to end the present processing as in the first embodiment.

As described above, the substance introduction device 1 (1A, 1B) according to the first embodiment and the second embodiment of the present disclosure is the substance introduction device 1 which introduces a substance into a cell by electroporation, and includes: one or more substance introduction units 2, the substance introduction unit 2 including the accommodation container 21 configured to accommodate a cell suspension containing the cell and the substance and the pair of electrodes 22 configured to apply a voltage to the cell suspension accommodated in the accommodation container 21; and the liquid delivery unit 4 configured to deliver a liquid to the accommodation container 21. The accommodation container 21 has the first opening 21B and the second opening 21C, and the liquid delivery unit 4 is configured to fill the accommodation container 21 with the cell suspension before voltage application through the first opening 21B and to discharge the cell suspension after the voltage application from the accommodation container 21 through the second opening 21C, and to fill the accommodation container 21 with a cleaning solution through the second opening 21C and to discharge the cleaning solution from the accommodation container 21 through the first opening 21B.

According to the substance introduction device 1 having such a configuration, the electroporation can be repeatedly executed in the same container without enlarging the container for execution of the electroporation, so that a total amount of the cell suspension to be treated can be increased without changing an electroporation execution condition. Further, a direction in which the cleaning solution flows inside the accommodation container 21 of the substance introduction unit 2 is set to be opposite to a direction in which the cell suspension flows in the accommodation container 21 of the substance introduction unit 2, so that the stains, such as the cell residues or air bubbles, are likely to be peeled off from the accommodation container 21, and an effect of cleaning the pair of electrodes 22 and the accommodation container 21 and the pair of electrodes 22 can be improved. Therefore, according to the substance introduction device 1 of the present disclosure, the substance introduction efficiency in the electroporation is less likely to decrease when the electroporation is repeatedly executed in the same container, and the usefulness of a technique of introducing the substance into the cell by the electroporation.

Although the present disclosure has been described with reference to the drawings and embodiment, it should be noted that those skilled in the art can make various modifications and corrections on the basis of the present disclosure. Therefore, it should be noted that these modifications and corrections fall within the scope of the present disclosure. For example, the function or the like included in each means, each step, or the like can be rearranged not to be logically inconsistent, and a plurality of means, steps, or the like can be combined into one or divided.

For example, it has been described that the cell-containing buffer solution and the substance-containing buffer solution are suspended in the pre-introduction bag 10C in the above-described embodiment. However, the cell-containing buffer solution and the substance-containing buffer solution may be suspended when flowing into the pre-introduction bag 10C in the liquid delivery path 9A. This makes it possible to efficiently suspend the cell-containing buffer solution and the substance-containing buffer solution.

Further, for example, it has been described that the electroporation and the cleaning of the substance introduction unit 2 are alternately executed the same number of times in the above-described embodiment. However, the number of times of the electroporation may be different from the number of times of the cleaning. For example, the cleaning may be executed once while the electroporation is repeated a plurality of times in accordance with the likelihood of generation of the cell residues in the electroporation. Thus, it is possible to optimize the substance introduction efficiency in the electroporation and the processing speed of the electroporation.

Further, for example, it has been described that the entire operation (substance introduction method) of the substance introduction device 1 is automatically executed under the control of the control unit 8 in the above-described embodiment. However, a part or whole of the operation of the substance introduction device 1 may be manually executed. For example, some or all of the opening and closing of the opening and closing unit 23 of the substance introduction unit 2, the voltage application with respect to the pair of electrodes 22 by the voltage application unit 3, the delivery by the liquid delivery unit 4, the counting of living cells by the cell counter 5, the switching of the flow path of the liquid delivery path 9 by the valve unit 7, and the like may be manually executed. Thus, a structure of the substance introduction device 1 can be simplified, and an increase in manufacturing cost of the substance introduction device 1 can be suppressed.

Further, for example, an embodiment in which a general-purpose computer is configured to function as the control unit 8 according to the above-described embodiment is also possible. Specifically, a program describing processing contents for implementing the respective functions of the control unit 8 according to the above-described embodiment is stored in a memory of the general-purpose computer, and the program is read and executed by a processor. Therefore, the present disclosure can also be implemented as the program executable by the processor or a non-transitory computer-readable medium that stores the program.

The present disclosure relates to a substance introduction device and a substance introduction method.

What is claimed is:

1. A substance introduction device, which introduces a substance into a cell by electroporation, comprising:
at least one substance introduction unit comprising an accommodation container configured to accommodate a cell suspension containing the cell and the substance and a pair of electrodes configured to apply a voltage to the cell suspension accommodated in the accommodation container; and
a liquid delivery unit configured to deliver a liquid to the accommodation container,
wherein the accommodation container has a first opening and a second opening, and
the liquid delivery unit is configured
to fill the accommodation container with the cell suspension before voltage application through the first opening and to discharge the cell suspension after the voltage application from the accommodation container through the second opening, and
to fill the accommodation container with a cleaning solution through the second opening and to discharge the cleaning solution from the accommodation container through the first opening.

2. The substance introduction device according to claim 1, wherein the at least one substance introduction unit includes an opening and closing unit configured to be capable of opening and closing the second opening of the accommodation container.

3. The substance introduction device according to claim 1, wherein the first opening is configured to be located vertically above the second opening.

4. The substance introduction device according to claim 1, wherein the at least one substance introduction unit comprises:
a plurality of substance introduction units connected in series,
wherein the plurality of substance introduction units are connected in series by linking a second opening of an accommodation container of a first substance introduction unit of the plurality of substance introduction units and a first opening of an accommodation container of a second substance introduction unit of the plurality of substance introduction units.

5. The substance introduction device according to claim 1, further comprising a filtering unit configured to remove a cell residue from the cell suspension before the voltage application with which the accommodation container is to be filled.

6. The substance introduction device according to claim 1, further comprising a filtering unit configured to remove a cell residue from the cell suspension after the voltage application discharged from the accommodation container.

7. The substance introduction device according to claim 1, further comprising a cell counter configured to count a living cell in the cell suspension before the voltage application with which the accommodation container is to be filled.

8. The substance introduction device according to claim 1, further comprising a cell counter configured to count a living cell in the cell suspension after the voltage application discharged from the accommodation container.

9. A substance introduction method for introducing a substance into a cell by electroporation, the substance introduction method comprising:
preparing at least one substance introduction unit comprising an accommodation container configured to accommodate a cell suspension containing the cell and the substance and a pair of electrodes configured to apply a voltage to the cell suspension accommodated in the accommodation container;
filling the accommodation container with the cell suspension before voltage application through a first opening of the accommodation container;
applying, by the pair of electrodes, a voltage to the cell suspension accommodated in the accommodation container;
discharging the cell suspension after the voltage application from the accommodation container through a second opening of the accommodation container;
filling the accommodation container with a cleaning solution through the second opening; and
discharging the cleaning solution from the accommodation container through the first opening.

10. The substance introduction method according to claim 9, wherein
the first opening is configured to be located vertically above the second opening,
the filling the accommodation container with the cleaning solution through the second opening includes filling the accommodation container with the cleaning solution from a lower side of the accommodation container through the second opening, and
the discharging the cleaning solution from the accommodation container through the second opening includes discharging the cleaning solution from an upper side of the accommodation container through the first opening.

11. The substance introduction method according to claim 9, further comprising removing a cell residue from the cell suspension before the voltage application with which the accommodation container is to be filled.

12. The substance introduction method according to claim 9, further comprising removing a cell residue from the cell suspension after the voltage application discharged from the accommodation container.

13. The substance introduction method according to claim 9, further comprising counting a living cell in the cell suspension before the voltage application with which the accommodation container is to be filled.

14. The substance introduction method according to claim 9, further comprising counting a living cell in the cell suspension after the voltage application discharged from the accommodation container.
